(19) United States
(12) Patent Application Publication
(10) Pub. No.: US 2024/0133281 A1
(43) Pub. Date: Apr. 25, 2024

(54) CALCULATION SYSTEMS FOR PREDICTING PROPPANT EMBEDDING DEPTH BASED ON SHALE SOFTENING EFFECT

(71) Applicant: SOUTHWEST PETROLEUM UNIVERSITY, Chengdu (CN)

(72) Inventors: Cong LU, Chengdu (CN); Qijun ZENG, Chengdu (CN); Jianchun GUO, Chengdu (CN); Jiaxing LIU, Chengdu (CN); Jun WU, Chengdu (CN); Junkai LU, Chengdu (CN); Cheng LUO, Chengdu (CN); Guangqing ZHOU, Chengdu (CN); Xianbo MENG, Chengdu (CN); Jiandong WANG, Chengdu (CN); Yanhui LIU, Chengdu (CN); Xiaoshan WANG, Chengdu (CN); Xin SHAN, Chengdu (CN)

(73) Assignee: SOUTHWEST PETROLEUM UNIVERSITY, Chengdu (CN)

(21) Appl. No.: 18/395,494

(22) Filed: Dec. 23, 2023

Related U.S. Application Data
(63) Continuation-in-part of application No. 18/301,205, filed on Apr. 14, 2023.

(30) Foreign Application Priority Data
Apr. 15, 2022 (CN) 202210393249.8

(51) Int. Cl. E21B 43/26; E21B 43/267; E21B 49/02; G01N 1/28; G01N 3/42; G01N 33/24; G06F 30/20

(52) U.S. Cl. CPC E21B 43/2607; E21B 43/267; E21B 49/02; G01N 1/28; G01N 3/42; G01N 33/24; G06F 30/20; E21B 2200/20

(57) ABSTRACT

A calculation system for predicting a proppant embedding depth based on a shale softening effect is provided, including a sampling test terminal, a scheduling module, a monitoring module, and a calculation module, wherein the scheduling module, the monitoring module, and the calculation module are connected in communication, and the monitoring module is connected to an external operating system through a wireless network, wherein the external operating system is configured to perform a hydraulic fracturing operation and receive a first control signal and/or a second control signal from the monitoring module. The sampling test terminal is configured to test the samples and obtain test data. The scheduling module is configured to determine a target construction parameter. The monitoring module is configured to: based on a difference between actual estimated proppant embedding volumes and a preset proppant embedding volume in the target operation area, issue a first warning message and/or send the first control signal to a fracturing control pump in the external operating system; in response to the characteristic parameters of the hydraulic fractures meeting the hydraulic fracture warning condition, issue a second warning information and/or send the second control signal to the fracturing control pump in the external operation system.

### CROSS REFERENCE TO RELATED APPLICATIONS

The present application is a continuation-in-part application of U.S. patent applicant Ser. No. 18/301,205, filed on Apr. 14, 2023, which claims priority of Chinese Patent Application No. 202210393249.8, filed on Apr. 15, 2022, the entire contents of which are incorporated herein by reference.

### TECHNICAL FIELD

The present disclosure relates to the technical field of oil and gas development, and in particular to a calculation system for predicting proppant embedding depth based on a shale softening effect.

### BACKGROUND

Shale gas has gradually become a hotspot in the exploration and development of unconventional natural gas in the world. However, shale reservoirs have poor physical properties such as porosity and permeability, and industrial production of shale gas is obtained by performing hydraulic fracturing on the shale reservoirs. At the same time, the fracturing fluid injected into the formation by hydraulic fracturing is difficult to drain out the formation after the construction is completed. A large amount of stranded fracturing fluids contacts the formation rock for a long time, and the shale fracture surfaces change in the mechanical properties under the action of high temperature and high pressure. Therefore, it is necessary to study and analyze the calculation system for proppant embedding depth under a softening effect of shale, which is of great significance for the optimization of fracture width formed by hydraulic fracturing.

Scholars at home and abroad have mainly established two types of models for proppant embedding, namely an empirical model and a theoretical model. The empirical model is to study the process of proppant embedded into fractures under formation conditions by carrying out propped fracture width tests, and then obtained based on test data and statistical theory; the theoretical model includes an analytical model and a numerical model. The analytical model is to describe the process of proppant embedded into the rock mass as a contact mechanics problem, establish a physical model of suitable dimensions based on the physical process of embedding, and calculate using Hooke's law and Hertz contact theory as the basis. With the development of numerical analysis software, scholars currently use software such as finite element and discrete element and carry out secondary development on the software to perform numerical calculations, and accurately calculate the stress conditions at any time and at any node of the 3D solid model and observe the deformation law of the contact surface directly. However, the existing numerical models generally ignore the influence of formation fluid for simulation, and the current hydraulic fracturing construction of shale gas wells is to inject thousands of cubic meters of fracturing fluid into the formation. If a calculation system for predicting proppant embedding depth based on a shale softening effect can be established, the calculation system will play a role in advancing the research on the width of artificial fractures produced by hydraulic fracturing of shale gas wells.

### SUMMARY

The technical solution provided by the present disclosure to solve the above technical problems is a calculation system for predicting a proppant embedding depth based on a shale softening effect. The calculation system comprises a sampling test terminal, a scheduling module, a monitoring module, and a calculation module. The scheduling module, the monitoring module, and the calculation module are connected in communication, and the monitoring module is connected to an external operating system through a wireless network. The external operating system is configured to perform a hydraulic fracturing operation and receive a first control signal and/or a second control signal from the monitoring module.

The sampling test terminal is configured to in response to receiving a sampling test instruction, take one or more samples from a rock layer, test the samples, and obtain test data.

The scheduling module is configured to obtain one or more sets of candidate construction parameters, the candidate construction parameters including candidate proppant parameters and candidate operation areas; obtain formation parameters of the candidate operation areas among the candidate construction parameters; based on the candidate construction parameters and the formation parameters of the candidate operation area, generate a calculation instruction, send the calculation instruction to the calculation module, and obtain predicted proppant embedding volumes corresponding to the candidate construction parameters from the calculation module; and determine a target construction parameter from the candidate construction parameters whose the predicted proppant embedding volumes are less than first preset thresholds.

The monitoring module is configured to obtain the target construction parameters for mining, and obtain the predicted proppant embedding volume corresponding to the target construction parameter from the calculation module; determine a hydraulic fracture warning condition during an operation period based on the predicted proppant embedded volume corresponding to the target construction parameter; for each preset cycle, determine characteristic parameters of one or more hydraulic fractures and rock quality features of one or more points in a target operation area based on second sensing data obtained at multiple times from one or more sensors deployed in the target operation area; determine, based on the rock quality features, actual estimated proppant embedding volumes corresponding to the rock quality features of one or more points in the target operation area through the test data under current proppant parameters; based on a difference between the actual estimated proppant embedding volumes and a preset proppant embedding volume in the target operation area, issue a first warning message and/or send the first control signal to a fracturing control pump in the external operating system, wherein the first control signal indicates that the fracturing control pump performs an adjustment on a particle size of proppant or a concentration of proppant of the fracturing fluid injected into a pipeline, and an adjustment amount of the adjustment in the first control signal is determined based on the difference between the actual estimated proppant embedding volumes and the preset proppant embedding volume; and in response to the characteristic parameters of the one or more hydraulic fractures meeting the hydraulic fracture warning condition, issue a second warning information and/or, send the second control signal to the fracturing control pump in the external operation system, wherein the second control signal indicates that the fracturing control pump performs an adjustment on the particle size of the proppant or the concentration of the proppant of fracturing fluid into the pipeline, and an adjustment amount of the adjustment in the second control signal is determined based on a difference between the characteristic parameters of the one or more hydraulic fractures and a threshold in the hydraulic fracture warning condition.

In some embodiments of the present disclosure, the calculation module includes a plurality of sub modules. The plurality of sub modules include a first determining module, a second determining module, a model module, a third determining module, and a fourth determining module. The first determining module is configured to determine a spontaneous imbibition depth-soaking time curve, wherein the curve is obtained by conducting a spontaneous imbibition test on faces of different standard cores at different soaking times respectively and utilizing a modified Lucas-Washburn (LW) model under the spontaneous imbibition effect, and the standard cores are obtained based on a target block shale. The second determining module is configured to determine a Young's modulus-soaking time curve of core surfaces, wherein the curve is obtained by drying the standard cores at different soaking times, conducting a nano-indentation test on surfaces of the standard cores respectively. The model module is configured to establish a 3D model of proppant embedded in a rock slab by a finite element manner, wherein the rock slab in the 3D model is divided into an unsoftened layer and a softened layer, and Young's modulus of the unsoftened layer is set as Young's modulus of standard cores; a thickness of the softened layer is set according to the spontaneous imbibition depth-soaking time curve, and Young's modulus of the softened layer is set according to the Young's modulus-soaking time curve, and a proppant embedding model containing the softened layer is obtained. The third determining module is configured to obtain an embedding volume-soaking time curve by performing numerical simulation on the proppant embedding model containing the softened layer with set parameters. The fourth determining module is configured to modify equivalent Young's modulus of proppant embedded in a rock mass based on the embedding volume-soaking time curve, and obtain a calculation formula for proppant embedding volume considering the softening effect;

$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{t} \} ,$$

$${E}_{t} = a {E}_{0} {e}^{- bt} .$$

Where w denotes a proppant embedding volume, a unit of which is millimeter (mm); a₀ and a₁ denote modification factors, which are 0.0646 and 18.2 respectively, and dimensionless; R denotes a particle size of proppant, a unit of which is mm; P denotes crustal stress, a unit of which is MegaPascal (MPa); E₁ denotes Young's modulus of the proppant, a unit of which is MPa; v₁ denotes a Poisson's ratio of the proppant, which is dimensionless; v₂ denotes a Poisson's ratio of the rock slab, which is dimensionless; H denotes a thickness of a rock slab, a unit of which is mm; t denotes a soaking time, a unit of which is day (d); E t denotes an equivalent Young's modulus, a unit of which is MPa; E₀ denotes Young's modulus of a standard core, a unit of which is MPa; and a and b denote fitting coefficients.

### BRIEF DESCRIPTION OF THE DRAWINGS

The present disclosure will be further illustrated by way of exemplary embodiments, which will be described in detail with the accompanying drawings. These embodiments are non-limiting, and in these embodiments, the same number indicates the same structure, wherein:

FIG. 1 is a module diagram illustrating an exemplary calculation system for predicting proppant embedding depth based on a shale softening effect according to some embodiments of the present disclosure;

FIG. 2 is a sub module diagram illustrating a calculation module according to some embodiments of the present disclosure;

FIG. 3 is a flowchart illustrating an exemplary process for determining a spontaneous imbibition depth-soaking time curve according to some embodiments of the present disclosure;

FIG. 4 is a schematic diagram illustrating an exemplary wetting angle prediction model according to some embodiments of the present disclosure;

FIG. 5 is a schematic diagram illustrating an exemplary determination of drying parameters according to some embodiments of the present disclosure;

FIG. 6 is a schematic diagram illustrating an exemplary established rock slab model containing a softened layer according to some embodiments of the present disclosure;

FIG. 7 is a schematic diagram illustrating an exemplary proppant embedding model containing a softened layer according to some embodiments of the present disclosure;

FIG. 8 is a curve illustrating a relationship between a softening depth and a soaking time according to some embodiments of the present disclosure;

FIG. 9 is a curve illustrating a relationship between Young's modulus and the soaking time according to some embodiments of the present disclosure;

FIG. 10 is a curve illustrating a relationship between a proppant embedding depth and a soaking time of a numerical model under different closure stress according to some embodiments of the present disclosure.

### DETAILED DESCRIPTION

In order to more clearly illustrate the technical solutions of the embodiments of the present disclosure, the following briefly introduces the drawings that need to be used in the description of the embodiments. Apparently, the accompanying drawings in the following description are only some examples or embodiments of the present disclosure, and those skilled in the art can also apply the present disclosure to other similar scenarios without creative effort. Unless obviously obtained from the context or the context illustrates otherwise, the same numeral in the drawings refers to the same structure or operation.

It should be understood that the words “system”, “device”, “unit” and/or “module” as used herein is a method for distinguishing different components, elements, parts, parts, or assemblies of different levels. However, the words may be replaced by other expressions if other words can achieve the same purpose.

As indicated in the present disclosure and claims, the terms “a”, “an”, and/or “the” are not specific to the singular and may include the plural unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

Flowcharts are used in the present disclosure to illustrate the operation performed according to the system of the embodiments of the present disclosure. It should be understood that the preceding or following operations are not necessarily performed in exact order. Instead, various steps may be processed in reverse order or simultaneously. At the same time, other operations can be added to these procedures, or a certain step or steps can be removed from these procedures.

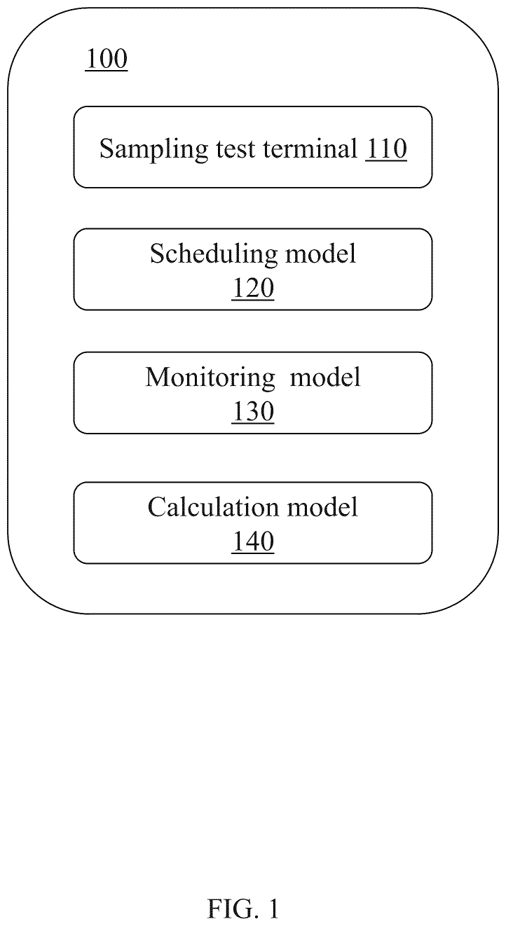

FIG. 1 is a module diagram illustrating an exemplary calculation system for predicting proppant embedding depth based on a shale softening effect according to some embodiments of the present disclosure. In some embodiments, the calculation system 100 for predicting the proppant embedding depth based on shale softening effect may include a sampling test terminal 110, a scheduling module 120, a monitoring module 130, and a calculation module 140. The scheduling module 120, the monitoring module 130, and the calculation module 140 may be connected in communication, the monitoring module 130 may be connected with an external operating system through a wireless network, and the external operating system may be configured to perform a hydraulic fracturing operation and receive a first control signal and/or a second control signal from the monitoring module 130.

In some embodiments, the sampling test terminal 110 may be configured to: in response to receiving a sampling test instruction, take one or more samples from the rock layer, test the samples, and obtain test data. For example, the sampling test terminal 110 may conduct a spontaneous imbibition test on faces of one or more samples at different soaking times, respectively.

In some embodiments, the scheduling module 120 is configured to obtain one or more sets of candidate construction parameters, the candidate construction parameters including candidate proppant parameters and candidate operation areas; obtain formation parameters of the candidate operation areas among the candidate construction parameters; generate a calculation instruction based on the candidate construction parameters and the formation parameters of the candidate operation area, send the calculation instruction to the calculation module 140, and obtain predicted proppant embedding volumes corresponding to the candidate construction parameters from the calculation module 140; and determine a target construction parameter from the candidate construction parameters whose the predicted proppant embedding volumes are less than first preset thresholds. More information about obtaining predicted proppant embedding volumes corresponding to the candidate construction parameters by the calculation module may be found in the relevant description of the present disclosure.

Candidate construction parameters refer to construction parameters available for selection. The construction parameters refer to parameters related to natural gas collection. In some embodiments, each set of candidate construction parameters includes candidate proppant parameters and candidate operation areas.

Candidate proppant parameters refer to the proppant parameters corresponding to the proppants available for selection. The proppant parameters refer to the parameters used to reflect the characteristics of the proppant. In some embodiments, the candidate proppant parameters include at least one of a particle size of the candidate proppant, a concentration of the candidate proppant, a Young's modulus of the candidate proppant, and a Poisson's ratio of the candidate proppant.

Candidate operation areas refer to operation areas available for selection. The operation areas refer to areas where natural gas extraction is carried out. For example, the operation areas include formation areas that cover a drilling path and surrounding.

In some embodiments, the scheduling module 120 may obtain one or more sets of candidate construction parameters by accessing the storage device. In some embodiments, the scheduling module 120 may obtain one or more sets of candidate construction parameters by user inputting.

Formation parameters refer to parameters related to formation characteristics. In some embodiments, formation parameters may include at least one of a thickness of a rock layer, a Poisson's ratio of the rock layer, and a Young's modulus of the rock layer.

In some embodiments, the scheduling module 120 may determine an average formation stress of the candidate operation areas based on first sensing data obtained at a plurality of times by one or more sensors deployed in the candidate operation areas; and generate a sampling test instruction, send the sampling test instruction to the sampling test terminal 110, and determine the formation parameters of the candidate operation areas based on the test data obtained from the sampling test terminal 110.

Sensors refer to measuring devices that is capable of converting nonelectrical quantity to corresponding electrical quantity or electrical parameter to output. For example, sensors may include pressure sensors, acoustic sensors, etc.

In some embodiments, sensors may be deployed in candidate operation areas at a preset depth, and a distance between adjacent sensors is the preset distance. For example, in the candidate operation area, holes with a preset depth may be drilled at a preset distance, and sensors may be deployed in the holes. The preset depth and the preset distance may be default values, preset values, etc.

In some embodiments of the present disclosure, by setting an interval distance between adjacent sensors as a preset distance, it may obtain more formation information and reduce resource waste.

The first sensing data refers to formation data before natural gas extraction. The formation data refers to data related to the formation characteristics of the candidate operation areas. For example, the first sensing data may include pressure data, acoustic data, etc.

The average formation stress refers to an average value of stress in the formation.

In some embodiments, for each candidate operation area, the scheduling module 120 may determine the formation stress at the corresponding position of each sensor based on the first sensing data obtained from each sensor at multiple times, calculate an average value of the formation stresses at the corresponding positions of all sensors, and determine the average value as the average formation stress of the candidate operation areas. The scheduling module 120 may determine the formation stress by a plurality of manners, such as a stress recovery manner, a geophysical manner, etc.

In some embodiments, the sampling test terminal 110 may determine the formation parameters by conducting a bending test on the sample and send the formation parameters to the scheduling module 120.

The first preset threshold refers to a threshold condition related to the predicted proppant embedded volume.

In some embodiments, different candidate construction parameters may correspond to different first preset thresholds. For example, the first preset threshold corresponding to candidate construction parameter A is A′, and the first preset threshold corresponding to candidate construction parameter B is B′.

In some embodiments, the first preset threshold may be a default value, a preset value, or the like.

In some embodiments, for each set of candidate construction parameters, the scheduling module 120 may determine the candidate operation area corresponding to the set of candidate construction parameter; and determine the first preset threshold corresponding to the set of candidate construction parameter based on a dispersion degree of formation characteristics of a plurality of points obtained by sensors in the candidate operation areas.

The dispersion degree of formation characteristics refers to a difference degree in formation characteristics corresponding to different points. The formation characteristics refer to parameters used to reflect the characteristics of the formation. For example, the formation characteristics may include formation stress.

The dispersion degree of formation characteristics may be represented by real numbers, the larger the real number, the greater the difference degree in formation characteristics corresponding to different points.

In some embodiments, for the candidate operation area corresponding to each set of candidate construction parameters, the scheduling module 120 may calculate a variance or a standard deviation of the formation characteristics corresponding to different points and determine the calculated variance or the calculated standard deviation as the dispersion degree of the formation characteristics corresponding to the candidate construction parameters.

In some embodiments, the greater the dispersion degree of the formation characteristics corresponding to the candidate construction parameters, the smaller the first preset thresholds corresponding to the candidate construction parameters.

In the candidate operation areas corresponding to the candidate construction parameters, the greater the dispersion degree of formation characteristics at multiple points obtained by multiple sensors, the greater the uncertainty of the formation areas. Therefore, when determining the target construction parameters, it is necessary to have strict screening conditions for candidate construction parameters and set small first preset thresholds, which is conducive to improving the usability of the final determined plan.

The target construction parameters refer to final determined construction parameters. In some embodiments, the target construction parameters include the target proppant parameters and the target operation areas. The target proppant parameters refer to the proppant parameters corresponding to the final determined proppants. The target operation areas refer to the final determined operation areas.

In some embodiments, the scheduling module 120 may arbitrarily select a set of candidate construction parameters as target construction parameters from the candidate construction parameters whose the predicted proppant embedding volumes are less than the first preset thresholds. In some embodiments, the scheduling module 120 may select the candidate construction parameter corresponding to a minimum predicted proppant embedding volume as the target construction parameter from the candidate construction parameters whose the predicted proppant embedding volumes are less than the first preset thresholds.

In some embodiments, the monitoring module 130 is configured to obtain the target construction parameters for currently mining, obtain the predicted proppant embedding volume corresponding to the target construction parameters from the calculation module 140, determine the hydraulic fracture warning condition during the operation period based on the predicted proppant embedded volumes corresponding to the target construction parameters.

In some embodiments, the monitoring module 130 may obtain target construction parameters by accessing scheduling module 120.

The hydraulic fracture warning condition refers to a triggering condition for hydraulic fracture warning. As an example, the hydraulic fracture warning condition may be monitoring a change amplitude of fracture widths of at least a preset number (e.g., 2) of hydraulic fractures to be greater than a second preset threshold (e.g., 2 cm) within a preset time interval (e.g., 2 seconds).

In some embodiments, the second preset threshold in the hydraulic fracture warning condition may be determined based on the predicted proppant embedding volume, the preset number of hydraulic fractures in the hydraulic fracture warning condition, and/or the preset time interval. For example, the smaller the predicted proppant embedded volume, the larger the second preset threshold. For example, the smaller the preset number of hydraulic fractures in the hydraulic fracture warning condition, the larger the second preset threshold. For example, the longer the preset time interval in the hydraulic fracture warning condition, the larger the second preset threshold.

In some embodiments, the monitoring module 130 may be configured to, for each preset cycle, determine the characteristic parameters of one or more hydraulic fractures and rock quality features of one or more points in a target operation area from the second sensing data obtained at multiple times from one or more sensors deployed in the target operation area; determine, based on the rock quality features, actual estimated proppant embedding volumes corresponding to the rock quality features of one or more points in the target operation area through the test data under current proppant parameters; based on a difference between the actual estimated proppant embedding volumes and a preset proppant embedding volume in the target operation area, issue a first warning message and/or send the first control signal to a fracturing control pump in the external operating system, the first control signal indicating that the fracturing control pump performs an adjustment on a particle size of proppant or a concentration of proppant of the fracturing fluid injected into a pipeline; in response to the characteristic parameters of hydraulic fractures meeting the hydraulic fracture warning condition, issue a second warning message, and/or send the second control signal to the fracturing control pump in the external operating system, the second control signal indicating that the fracturing control pump performs an adjustment on the particle size of the proppant or the concentration of the proppant of the fracturing fluid into the pipeline.

In some embodiments, the preset cycle may be a default value, a preset value, or the like.

In some embodiments, the preset cycle is related to at least one of a size of the target operation area, a total depth of the formation, and a current remaining available computing resource. For example, the larger the size of the target operation area, the smaller the preset cycle. For example, the larger the total depth of the formation in the target operation area, the smaller the preset cycle. For example, the larger the current remaining available computing resource, the smaller the preset cycle.

The current remaining available computing resource refers to the current idle computing resource.

The larger the size of the target operation area and the deeper the total depth of the formation, the greater the uncertainty of the formation area. Therefore, the preset cycle may be set to be smaller to be more sensitive to obtain formation information, so as to timely detect construction problems to improve construction safety. The larger the current available computing resource, the smaller the preset cycle may be set, which not only fully utilizes computing resource but also improves construction safety.

In some embodiments, one or more sensors may also include electromagnetic wave sensors. The second sensing data refers to the formation data during the extraction of natural gas. For example, the second sensing data may include pressure data of the target operation area and electromagnetic wave data of the target operation area, etc.

The characteristic parameters of hydraulic fractures refer to the parameters used to reflect the morphology of hydraulic fractures. For example, the characteristic parameters of hydraulic fractures include at least one of the length, width, and effective support area of hydraulic fractures.

In some embodiments, the monitoring module 130 may determine the characteristic parameters of hydraulic fractures based on the second sensing data through manners such as a finite element manner, a boundary element manner, etc.

The rock quality feature refers to a feature associated with the standard core. For example, the rock quality feature may include but is not limited to, a rock quality type (e.g., conglomerate, glutenite, sandstone), a rock pore type, a rock surface porosity, a rock permeability, a rock density, etc. of the standard cores.

In some embodiments, the monitoring module 130 may determine the rock quality features of one or more points within the target operation area based on the second sensing data through a rock quality feature model. The rock quality feature model is a machine learning model, such as the Deep Neural Network (DNN) model.

In some embodiments, the rock quality feature model may be trained through multiple second training samples with second labels. For example, multiple second training samples may be input into an initial rock quality feature model, and a loss function may be constructed based on the second labels and the outputs of the initial rock quality feature model. The parameters of the initial rock quality feature model may be iteratively updated based on the loss function. When an iteration preset condition is met, a model training is completed, and a trained rock quality feature model is obtained. The iteration preset condition may be convergence of the loss function, a count of iterations reaching a threshold, etc.

In some embodiments, the second training samples may include sample second sensing data of multiple sample points in the sample target operation area; and the second labels may include the sample rock quality features corresponding to the second training samples. In some embodiments, the second training samples and corresponding second labels may be obtained based on historical data.

In some embodiments, a corresponding relationship between different rock quality features and proppant embedding volumes may be stored in the storage device in advance based on completed experimental data. The monitoring module 130 may access the storage device based on the determined rock quality features of the target operation area and determine the actual estimated proppant embedding volumes corresponding to the rock quality features under the current proppant parameters through the corresponding relationship.

In some embodiments, the monitoring module 130 may calculate the difference between the actual estimated proppant embedding volume and the preset proppant embedding volume and issue a first warning message in response to the difference exceeding a difference threshold.

In some embodiments, the monitoring module 130 may issue the first warning information in various ways. For example, the monitoring module 130 may issue the first warning information through voice broadcasting. For example, the monitoring module 130 may issue the first warning messages through red lights.

In some embodiments, when the first control signal indicates that the fracturing control pump performs an adjustment on the particle size of proppant and/or the concentration of proppant of the fracturing fluid injected into the pipeline, an adjustment amount of the adjustment is determined based on the difference between the actual estimated proppant embedding volume and the preset proppant embedding volume. For example, the greater the difference between the actual estimated proppant embedding volume and the preset proppant embedding volume, the greater the adjustment amount.

In some embodiments, the warning manner of the second warning information may be the same or different from the warning manner of the first warning information. For example, the first warning information may be issued through red lights, the second warning information may be issued through blue lights.

In some embodiments, when the second control signal indicates that the fracturing control pump performs an adjustment on the particle size of the proppant and the concentration of the proppant of fracturing fluid into the pipeline, and an adjustment amount of the adjustment is determined based on the difference between the characteristic parameters of hydraulic fractures and the threshold in the hydraulic fracture warning condition.

As an example only, in response to the characteristic parameters of hydraulic fractures meeting the hydraulic fracture warning condition, the monitoring module 130 may calculate the difference between the characteristic parameters of hydraulic fractures and the threshold in the hydraulic fracture warning condition. The larger the difference, the greater the adjustment amount.

It should be noted that the above descriptions of the system and its modules are only for the convenience of description and do not limit the present disclosure to the scope of the illustrated embodiments. It should be understood that for those skilled in the art, after understanding the principles of the system, it is possible to combine various modules arbitrarily or form a subsystem to connect with other modules without departing from the principles. In some embodiments, the sampling test terminal 110, the scheduling module 120, the monitoring module 130, the calculation module 140 disclosed in FIG. 1 may be different modules in one system, or one module can realize the functions of the above-mentioned two or more modules. For example, each module may share one storage module, or each module may have its own storage module. Such deformations are within the protection scope of the present disclosure.

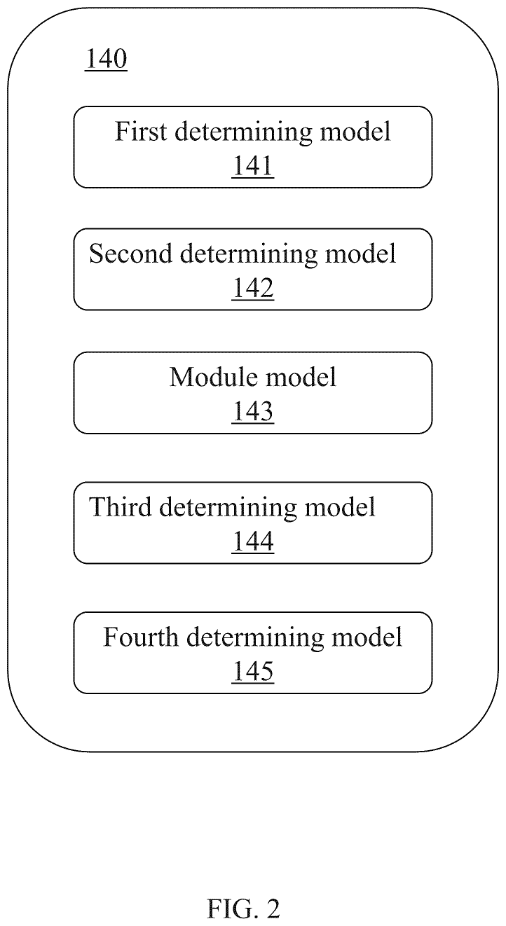

FIG. 2 is a sub module diagram illustrating a calculation module according to some embodiments of the present disclosure. In some embodiments, the calculation module 140 may include a first determining module 141, a second determining module 142, a model module 143, a third determining module 144 and a fourth determining module 145.

The first determining module 141 may be configured to determine a spontaneous imbibition depth-soaking time curve, wherein the curve is obtained by conducting a spontaneous imbibition test on faces of different standard cores at different soaking times respectively and utilizing a modified Lucas—Washburn (LW) model under the spontaneous imbibition effect, and the standard cores are obtained based on a target block shale.

The first determining module 141 may be further configured to determine a first soaking time set; determine first wetting angles of the standard cores, the first wetting angles including wetting angles of the standard cores corresponding to the at least one soaking time in the first soaking time set; predict second wetting angles of the standard cores based on the first wetting angles; the second wetting angles including wetting angles of the standard cores corresponding to the least one soaking time in a second soaking time set; obtain the spontaneous imbibition depth-soaking time curve by utilizing the modified LW model under the spontaneous imbibition effect based on the first wetting angles and the second wetting angles.

The first determining module 141 may be further configured to determine the second wetting angle of the standard core through processing the first wetting angles by a wetting angle prediction model, and the wetting angle prediction model is a machine learning model.

The first determining module 141 may be further configured to obtain no less than a preset count of training samples, the training samples including sample rock quality features of sample standard cores, a sample first soaking time set, first sample wetting angles corresponding to the sample first soaking time set, a sample second soaking time set, and a liquid type of sample liquid for soaking sample standard cores; and iteratively update an initial wetting angle prediction model by utilizing the plurality of training samples to obtain the wetting angle prediction model.

The first determining module 141 may be further configured to determine a corresponding equivalent soaking condition based on the soaking time in the first soaking time set, conduct a soaking test on the standard core with the equivalent soaking condition, and determine wetting angles obtained from a test result as the first wetting angles of the standard cores.

The second determining module 142 may be configured to determine a Young's modulus-soaking time curve of core surfaces, wherein the curve is obtained by drying the standard cores at different soaking times and conducting a nano-indentation test on surfaces of the standard cores respectively.

The second determining module 142 may be further configured to determine the drying parameters of the standard cores after the soaking times based on the rock quality features and soaking time of the standard cores.

The model module 143 may be configured to establish a 3D model of proppant embedded in a rock slab by a finite element manner, wherein the rock slab in the 3D model is divided into an unsoftened layer and a softened layer, and Young's modulus of the unsoftened layer is set as Young's modulus of the standard core; a thickness of the softened layer is set according to the spontaneous imbibition depth-soaking time curve, and Young's modulus of the softened layer is set according to the Young's modulus-soaking time curve, and a proppant embedding model containing the softened layer is obtained.

The third determining module 144 may be configured to obtain an embedding volume soaking time curve by performing numerical simulation on the proppant embedding model containing the softened layer with set parameters.

The third determining module 144 may be further configured to set simulated parameters of the proppant embedding model containing the softened layer respectively according to the different soaking times; apply closure stress to an upper slab of the 3D model by utilizing a stress interaction effect, completely fix a lower slab of the 3D model to simulate a crustal fracture closure process, output an average embedding volume of the upper slab and lower slab after the model is stabilized, and obtain the embedding volume-soaking time curve at the different soaking times.

The fourth determining module 145 may be configured to modify equivalent Young's modulus of proppant embedded in a rock mass based on the embedding volume-soaking time curve, and obtain a calculation formula for a proppant embedding volume considering the softening effect:

$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{t} \} ,$$

$${E}_{t} = a {E}_{0} {e}^{- bt} .$$

Where w denotes a proppant embedding volume, a unit of which is millimeter (mm); a₀ and a₁ denote modification factors, which are 0.0646 and 18.2 respectively, and dimensionless; R denotes a particle size of proppant, a unit of which is mm; P denotes crustal stress, a unit of which is MegaPascal (MPa); E₁ denotes Young's modulus of proppant, a unit of which is MPa; v₁ denotes a Poisson's ratio of the proppant, which is dimensionless; v₂ denotes a Poisson's ratio of the rock slab, which is dimensionless; H denotes thickness of a rock slab, a unit of which is mm; t denotes a soaking time, a unit of which is day (d); Eₜ denotes an equivalent Young's modulus, a unit of which is MPa; E₀ denotes Young's modulus of a standard core, a unit of which is MPa; and a and b denote fitting coefficients.

The fourth determining module 145 may be further configured to (1) obtain an embedding volume at a soaking time t₁ according to the embedding volume-soaking time curve obtained by the numerical simulation, and introduce the embedding volume into the calculation formula for the proppant embedding volume of the proppant embedded in a rock mass, and obtain equivalent Young's modulus Eₜ₁ at the soaking time t₁ by calculating reversely, repeating the above process, obtaining equivalent Young's modulus Eₜ₂ at a soaking time t₂, equivalent Young's modulus Eₜ₃ at a soaking time t₃, . . . , equivalent Young's modulus Eₜₙ at a soaking time tₙ; and (2) according to equivalent Young's modulus corresponding to the different soaking times, obtain equivalent Young's modulus of a softened rock slab by regression.

In some embodiments, in response to receiving the calculation instruction, the formation parameters are input into the calculation formula for the proppant embedding volume to obtain the predicted proppant embedding volume. The calculation formula for the proppant embedding volume may be determined by a process including the following steps.

Step S1, determining a spontaneous imbibition depth-soaking time curve, wherein the curve is obtained by conducting a spontaneous imbibition test on faces of different standard cores at different soaking times respectively and utilizing a modified LW model under a spontaneous imbibition effect, and the standard cores are obtained based on a target block shale; wherein a relationship formula of the modified LW model under the spontaneous imbibition effect is:

h(t)=\sqrt{(rδγt cos θ)/2 τμ}.

Where h(t) denotes a spontaneous imbibition distance, a unit of which is m; t denotes the soaking time, a unit of which is second (s); r denotes an equivalent capillary radius, a unit of which is meter (m); γ denotes a fluid interfacial tension, a unit of which is Newton/meter (N/m); δ denotes a pore-shape factor, which is dimensionless; θ denotes a wetting angle, a unit of which is °; τ denotes a pore tortuosity, which is dimensionless; and μ denotes a fluid viscosity, a unit of which is Pascal second (Pa·s).

For example, after soaking standard cores with different core quality features under 0, 3, 5, 7, 15 days respectively, equivalent capillary radius, fluid interfacial tension, pore-shape factors, wetting angles, pore tortuosity, fluid viscosity of the standard cores with different quality features are obtained at different soaking times, and then a spontaneous imbibition distance of the standard cores soaked for different soaking times are obtained through a relationship formula of the modified LW model under the spontaneous imbibition effect.

Step S2, determining a Young's modulus-soaking time curve of core surfaces, wherein the curve is obtained by drying the standard cores at different soaking times and conducting a nano-indentation test on surfaces of the standard cores respectively. For example, the Young's modulus—soak time curve of the core surfaces is obtained by pressing an indenter into a standard core under an external load and measuring the magnitude of the load and the depth of pressing into the standard core.

Step S3, establishing a 3D model of proppant embedded in a rock slab by a finite element manner, and setting the following assumptions.
(1) The proppant is embedded in a fracture under closure stress, regardless of a situation of proppant fragmentation.
(2) The proppant has ideal sphericity and good sorting property, and the proppant is laid evenly in fractures and arranged regularly.
(3) An unsoftened layer of a rock slab is a homogeneous material, and a softened layer of the rock slab is a heterogeneous material, which is evenly divided according to the grid; wherein the softened layer refers to a rock layer softened after soaking; and the unsoftened layer refers to a rock layer that is not softened after soaking. (
4) A process of proppant embedding is considered elastic; and the constitutive equation is:

$$\{ σ \} = {[ D ]}_{e} \{ ε \}$$

$${[ D ]}_{e} = \frac{E ( 1 - ν )}{( 1 + ν ) ( 1 - 2 ν )} \vert \begin{matrix}\begin{matrix}1 & \frac{ν}{1 - ν} & \frac{ν}{1 - ν} \\ \frac{ν}{1 - ν} & 1 & \frac{ν}{1 - ν} \\ \frac{ν}{1 - ν} & \frac{ν}{1 - ν} & 1\end{matrix} & \begin{matrix}0 & 0 & 0 \\ 0 & 0 & 0 \\ 0 & 0 & 0\end{matrix} \\ \begin{matrix}0 & 0 & 0 \\ 0 & 0 & 0 \\ 0 & 0 & 0\end{matrix} & \begin{matrix}\frac{1 - 2 ν}{2 ( 1 - ν )} & 0 & 0 \\ 0 & \frac{1 - 2 ν}{2 ( 1 - ν )} & 0 \\ 0 & 0 & \frac{1 - 2 ν}{2 ( 1 - ν )}\end{matrix}\end{matrix} \vert .$$

Where [D]ₑ denotes a 3D elastic matrix; {σ} denotes an element stress matrix; {ϵ} denotes an element strain matrix; E denotes Young's modulus of a material, a unit of which is MPa; v denotes a Poisson's ratio of the material, which is dimensionless.

The rock slab includes an unsoftened layer and a softened layer respectively, wherein H_{soften} denotes a thickness of the softened layer, which is spontaneous imbibition depth; H_{unsoften} denotes a thickness of the unsoftened layer, which is calculated by a formula:

H_{unsoften}=H−H_{soften}.

Where H_{unsoften} denotes the thickness of the unsoftened layer, a unit of which is mm; H_{soften} denotes the thickness of the softened layer, a unit of which is mm; H denotes an overall thickness of the rock slab, a unit of which is mm; Young's modulus of the unsoftened layer is set as Young's modulus of the standard cores; the thickness of the softened layer is set according to the spontaneous imbibition depth-soaking time curve, and Young's modulus of the softened layer is set according to the Young's modulus-soaking time curve.

Step S4, obtaining an embedding volume-soaking time curve by performing numerical simulation on the proppant embedding model containing the softened layer with set parameters, including setting simulated parameters of the proppant embedding model containing the softened layer respectively according to the different soaking times; applying closure stress to an upper slab of the 3D model by utilizing a stress interaction effect, completely fixing a lower slab of the 3D model to simulate a crustal fracture closure process, outputting an average embedding volume of the upper slab and lower slab after the model is stabilized, and obtaining the embedding volume-soaking time curve at the different soaking times.

Step S5, modifying equivalent Young's modulus of proppant embedded in a rock mass based on the embedding volume-soaking time curve, and obtaining a calculation formula for a proppant embedding volume considering the softening effect.

$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{t} \}$$

$${E}_{t} = a {E}_{0} {e}^{- bt}$$

Where w denotes a proppant embedding volume, a unit of which is mm; a₀ and a₁ denote modification factors, which are 0.0646 and 18.2 respectively, and dimensionless; R denotes a particle size of proppant, a unit of which is mm; P denotes crustal stress, a unit of which is MPa; E₁ denotes Young's modulus of proppant, a unit of which is MPa; v₁ denotes a Poisson's ratio of the proppant, which is dimensionless; v₂ denotes a Poisson's ratio of the rock slab, which is dimensionless; H denotes a thickness of a rock slab, a unit of which is mm; t denotes a soaking time, a unit of which is d; Eₜ denotes an equivalent Young's modulus, a unit of which is MPa; E₀ denotes Young's modulus of a standard core, a unit of which is MPa; and a and b denote fitting coefficients.

The modifying equivalent Young's modulus of proppant embedded in a rock mass based on the embedding volume-soaking time curve includes the following steps.
(1) Obtaining a embedding volume at a soaking time ti according to the embedding volume-soaking time curve obtained by the numerical simulation, and introducing the embedding volume into the calculation formula for the proppant embedding volume of the proppant embedded in a rock mass, and obtaining equivalent Young's modulus Eₜ₁ at the soaking time t₁ by calculating reversely, repeating the above process, obtaining equivalent Young's modulus Eₜ₂ at a soaking time t₂, equivalent Young's modulus Eₜ₃ at a soaking time t₃, . . . , equivalent Young's modulus Eₜₙ at a soaking time tₙ.
(2) According to equivalent Young's modulus corresponding to the different soaking times, obtaining equivalent Young's modulus of a softened rock slab by regression.

In some embodiments, application scenarios of the calculation system for predicting proppant embedding depth based on a shale softening effect may include the calculation module, a storage device, network, and a terminal device, etc. The storage device may be configured to store data and/or instructions, for example, the storage device may store measurement data obtained by the sampling test terminal from a test, etc. The network may connect components of the system and/or connect the system to external resource components, allowing communication between the components, and communication with other components outside the system, facilitating the exchange of data and/or information. For example, the calculation module may obtain the spontaneous imbibition depth-soaking time curve from the storage device through the network. The calculation module may determine the spontaneous imbibition depth-soaking time curve and the Young's modulus-soaking time curve of core surfaces based on test data and transfer the spontaneous imbibition depth-soaking time curve and the Young's modulus-soaking time curve of core surfaces to the terminal device through the network. The terminal device may be one or more terminals used by a user. For example, the terminal device may include a mobile device, a computer laptop, a tablet, etc. or any combination thereof. The user of the terminal device may be an owner of the terminal device, and the user may view the spontaneous imbibition depth-soaking time curve and the Young's modulus-soaking time curve of core surfaces, etc. based on the terminal device.

In some embodiments of the present disclosure, automatically obtaining measurement data of a test, determining the spontaneous imbibition depth-soaking time curve, the Young's modulus-soaking time curve of core surfaces, establishing a 3D model, and finally obtaining the calculation formula for a proppant embedding volume considering the softening effect by the calculation system for predicting proppant embedding depth based on a shale softening effect may not only process a large amount of data, make parameters of obtained curves and calculation formulas more accurate, and also enhance calculation speed efficiently and shorten calculation time.

In some embodiments of the present disclosure, assumptions considered in numerical models in the present disclosure are closer to reality, simulated fracture width values under different softening conditions have a high fit with actual measured values, and the numerical models are strongly process-oriented, which can predict a fracture width value of an actual shale reservoir after hydraulic fracturing accurately and efficiently.

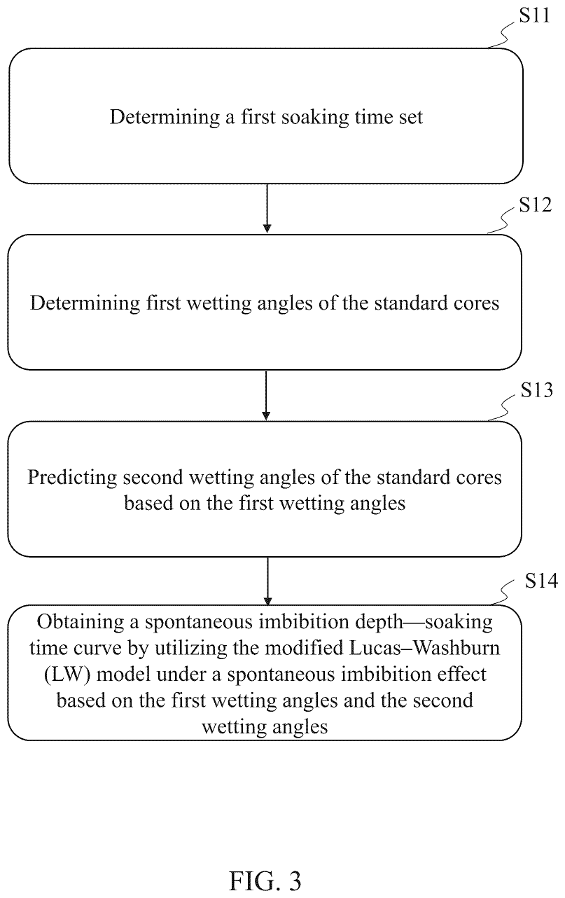

FIG. 3 is a flowchart illustrating an exemplary process for determining a spontaneous imbibition depth-soaking time curve according to some embodiments of the present disclosure. In some embodiments, step S1 may be executed based on a calculation module. As shown in FIG. 3, the step S1 includes the following steps.

Step S11, determining a first soaking time set.

The first soaking time set refers to a set of a first set of soaking times. For example, the first soaking time set may be represented as (0, 3, 5, 7, 9, 11). A count of soaking times in the first soaking time set may be more, such as ten.

In some embodiments, the calculation module may determine the first soaking time set in a plurality of ways. For example, the first soaking time set may be a default value, a preset value, or the like.

step S12, determining first wetting angles of the standard cores.

In some embodiments, the first wetting angles include wetting angles of the standard cores corresponding to the at least one soaking time in the first soaking time set. In some embodiments, the first wetting angles may be obtained by performing a test on the standard cores at soaking times in the first soaking time set respectively and calculating a test result. For more details about the wetting angle, please refer to step S1 and related descriptions.

In some embodiments, when performing a test on the standard cores at soaking times in the first soaking time set respectively, a corresponding equivalent soaking condition may be determined based on the soaking times in the first soaking time set, then a soaking test may be conducted on the standard core with the equivalent soaking condition, and wetting angles obtained from a test result may be determined as the first wetting angles of the standard cores.

The equivalent soaking condition refers to a soaking condition that has an equivalent soaking effect compared to effect at the soaking times in the first soaking time set. In some embodiments, a soaking time of the equivalent soaking condition is less than a soaking time of the first soaking time set. For example, if a soaking time of the first soaking time set is 5 days, the equivalent soaking condition may include a soaking time of 1 day, heating at a° C., and pressurization at bPa.

In some embodiments, the calculation module may determine the equivalent soaking condition in a plurality of ways. For example, the calculation module may process the soaking time through an equivalent soaking condition determination model to determine the equivalent soaking condition.

The equivalent soaking condition determination model may be a machine learning model for determining the equivalent soaking condition. The equivalent soaking condition determination model may be a Neural Networks (NN) model or other models. For example, a Recurrent Neural Network (RNN) model, etc.

In some embodiments, an input of the equivalent soaking condition determination model may include soaking times and rock quality features in the first soaking time set; an output of the equivalent soaking condition determination model may include the equivalent soaking condition.

The rock quality feature refers to a feature associated with the standard core. For example, the rock quality feature may include but is not limited to, a rock quality type (e.g., conglomerate, glutenite, sandstone), a rock pore type, a rock surface porosity, a rock permeability, a rock density, etc. of the standard cores.

In some embodiments, the equivalent soaking condition determination model may be obtained by training based on a plurality of first training samples with a first label. For example, the plurality of first training samples with a first label may be input into an initial equivalent soaking condition determination model, a value of a loss function is constructed by the first label and a result of an initial equivalent soaking condition determination model, and parameters of the model are determined by iteratively updating the initial equivalent soaking condition based on the loss function. When the loss function of the initial equivalent soaking condition determination model meets a preset iteration condition, the model training is completed, and the trained equivalent soaking condition determination model is obtained. The preset iteration condition may be that the loss function converges, a count of iterations reaches a threshold, or the like. For example, the count of iterations may be not less than a preset count, such as not less than 100,000 times.

In some embodiments, the first training samples may include sample rock quality features and a sample soaking time of the sample standard cores when tests are performed on different sample standard cores. The first label may include a sample equivalent soaking condition of the sample standard cores corresponding to the first training samples. For example, a count of the first training samples and the first label corresponding to the first training samples may be not less than a preset count of sets, such as not less than 100,000 sets. In some embodiments, the first training samples may be obtained based on historical data (for example, historical rock quality features and historical soaking times of standard cores), and the first label may be determined in the following manner.

First, a sample soaking test is conducted on the sample standard cores at the sample soaking time to obtain a sample test result x₀ of the sample soaking test (for example, the sample test result may include a wetting angle, Young's modulus); a plurality of sets of equivalent soaking conditions are generated (for example, randomly generate) based on the sample soaking time, and a test is conducted on the sample standard cores in a simulated environment (for example, using simulation software to simulate) based on the plurality of sets of equivalent soaking conditions to obtain test results under each equivalent soaking condition x₁, x₂, . . . , xₙ respectively, and an equivalent soaking condition corresponding to a test result with the largest similarity (for example, a test result xₖ) is obtained by calculating a similarity between each of the test results x₁, x₂, . . . , xₙ and a sample test result x₀ respectively.

At the same time, in a real environment, a soaking test is conducted on the standard cores under an equivalent soaking condition corresponding to the test result xₖ to obtain a test result xₖ′; and a similarity between the test result xₖ′ and the sample test result x₀ is calculated. If the similarity is greater than a similarity threshold, the equivalent soaking condition corresponding to the test result xₖ is determined as the first label of the first training samples. If the similarity is less than the similarity threshold, then a soaking test is conducted under equivalent soaking conditions corresponding to other test results in other simulated environments. If an equivalent soaking condition whose similarity is greater than the similarity threshold cannot be obtained in the end, the first label of the first training samples is marked as “original soaking time, heating=0° C., pressurization=0 Pa”. The similarity threshold may be an experience value, a default value, a preset value, or the like.

In some embodiments, the first training samples used for training the equivalent soaking condition determination model may include first training data and second training data. The first training data refers to the samples and labels composed of rock cores sampled in any candidate operation area. The second training data refers to the samples and labels composed of rock cores sampled from non-candidate operation areas.

In some embodiments, a ratio of the data amount between the first training data and the second training data in the first training samples is a preset ratio. In some embodiments, the preset ratio is related to the dispersion degree of formation characteristics in each candidate operation area. For example, the greater the dispersion degree of formation characteristics in each candidate operation area, the larger the preset ratio. For more information on the dispersion degree of formation characteristics, please refer to the relevant descriptions mentioned earlier.

In some embodiments of the present disclosure, priority is given to selecting rock cores from candidate operation areas to construct training data (i.e., the first training data) for training the equivalent soaking condition determination model, so that the model can prioritize learning more about the rock condition around the area where natural gas collection is to be carried out. However, if only the cores from candidate operation areas are used for model training, it may lead to overfitting, so a second training data consisting of cores from non-candidate operation areas is still needed. In addition, the preset ratio of the first training data and the second training data may be dynamically controlled based on the dispersion degree of formation characteristics. If the candidate operation areas are already covered with rich rock layer/rock quality with various characteristics, the ratio of the first training data may be larger and not lead to overfitting.

In some embodiments, the calculation module may construct a first target vector based on the soaking time and the rock quality feature; determine a first correlation vector through a first vector database based on the first target vector; and determine a reference equivalent soaking condition corresponding to the first correlation vector as an equivalent soaking condition corresponding to the first target vector.

The first target vector refers to a vector constructed based on the soaking time and the rock quality feature. There are many ways to construct the first target vector. For example, the calculation module may input the soaking time and rock quality feature into an embedding layer for processing to obtain the first target vector. In some embodiments, the embedding layer may be obtained through joint training with the wetting angle prediction model.

The first vector database includes a plurality of first reference vectors, and each of the plurality of first reference vectors has a corresponding reference equivalent soaking condition. For example, a count of the first reference vector in the first vector database may be not less than a preset count, such as not less than 100,000.

The first reference vector refers to a vector constructed based on a historical soaking time and historical rock quality feature when the test is performed on the standard cores during a historical time period, the reference equivalent soaking condition corresponding to the first reference vector may be a historical equivalent soaking condition when the test is performed on the standard core during a historical time period. For a construction manner of the first reference vector, please refer to the above construction manner of the first target vector.

In some embodiments, the calculation module may calculate a vector distance between the first target vector and the first reference vector and determine the equivalent soaking condition of the first target vector. For example, the calculation module may determine a first reference vector whose vector distance from the first target vector meets a preset condition as a first correlation vector, and determine a reference equivalent soaking condition corresponding to the first correlation vector as the equivalent soaking condition corresponding to the first target vector. The preset condition may be set according to situations. For example, the preset condition may be that the vector distance is the smallest or the vector distance is smaller than a distance threshold, or the like. The vector distance may include but is not limited to, cosine distances, Mahalanobis distances, Euclidean distances, or the like.

In some embodiments, the calculation module may also match the first target vector with the first reference vector in the first vector database in other ways. For example, the calculation module may perform vector matching by means of Nearest Neighbor Search (NN), Approximate Nearest Neighbor Search (ANN), or the like.

In some embodiments, the calculation module may determine the equivalent soaking condition based on the soaking time and the rock quality feature, and upload the equivalent soaking condition to the storage device for storage or transfer the equivalent soaking condition to the terminal device to display to the user through the network.

In some embodiments of the present disclosure, the calculation module may determine the equivalent soaking condition through the soaking time and the rock quality feature, perform simulation test under a large number of equivalent soaking conditions to obtain an equivalent soaking condition with the shortest soaking time and the closest soaking effect to a real environment, and conduct a soaking test on the standard core using the equivalent soaking condition instead of the soaking time, test time can be significantly shortened and test efficiency can be increased while obtaining a same test result.

Step S13, predicting second wetting angles of the standard cores based on the first wetting angles.

In some embodiments, the second wetting angles includes wetting angles of the standard cores corresponding to the least one soaking time in a second soaking time set.

The second soaking time set refers to a set of a second set of soaking times.

In some embodiments, the calculation module may determine the second soaking time set in a plurality of ways. For example, the second soaking time set may be a default value, a preset value, or the like.

In some embodiments, a time difference between the at least two soaking times in the first soaking time set may be greater than a preset threshold, and part of or all soaking times in the second soaking time set may be interpolated among the soaking times of the first soaking time set. The preset threshold may be an experience value, a default value, a preset value, or the like. In some embodiments, a count of soaking times in the first soaking time set may be greater than a count of soaking times in the second soaking time set. For example, the first soaking time set may be (0, 3, 5, 7, 9, 11), and an optional second soaking time set may be (1, 4, 6, 8).

In some embodiments of the present disclosure, by interpolating the soaking times in the second soaking time set among the first soaking time set, and the count of soaking times in the first soaking time set being greater than the count of soaking times in the second soaking time set, a majority of time point data is used to predict a minority of time point data, which is conducive to improving the accuracy of the second wetting angles in subsequent predictions.

In some embodiments, the calculation module may predict the second wetting angles of the standard cores based on the first wetting angles in a plurality of ways. For example, the calculation module may fit each soaking time in the first soaking time set and first wetting angles corresponding to each soaking time to obtain a fitting function, and determine the second wetting angles by the fitting function based on the soaking times in the second soaking time set.

In some embodiments, the calculation module may construct a second target vector based on the rock quality feature and the soaking time; determine a second correlation vector through a second vector database based on the second target vector; and determine a reference wetting angle corresponding to the second correlation vector as the second wetting angle corresponding to the second target vector.

The second target vector refers to a vector constructed based on the rock quality feature and soaking time. For a construction manner of the second target vector, please refer to the above-mentioned construction manner of the first target vector.

The second vector database includes a plurality of second reference vectors, and each second reference vector of the plurality of second reference vectors has a corresponding reference wetting angle. For example, a count of second reference vectors in the second vector database may be not less than a preset count, such as not less than 100,000.

The second reference vector refers to a vector constructed based on a historical rock quality feature and a historical soaking time when the test is performed on the standard core during a historical time period. The reference wetting angle corresponding to the second reference vector may be a historical wetting angle when the test is performed on the standard core during a historical time period. For a construction manner of the second reference vector, please refer to the above-mentioned construction manner of the first target vector.

In some embodiments, the calculation module may calculate a vector distance between the second target vector and the second reference vector respectively, and determine the second wetting angle of the second target vector. For a manner of determining the second wetting angle of the second target vector, please refer to the above-mentioned manner of determining the equivalent soaking condition of the first target vector.

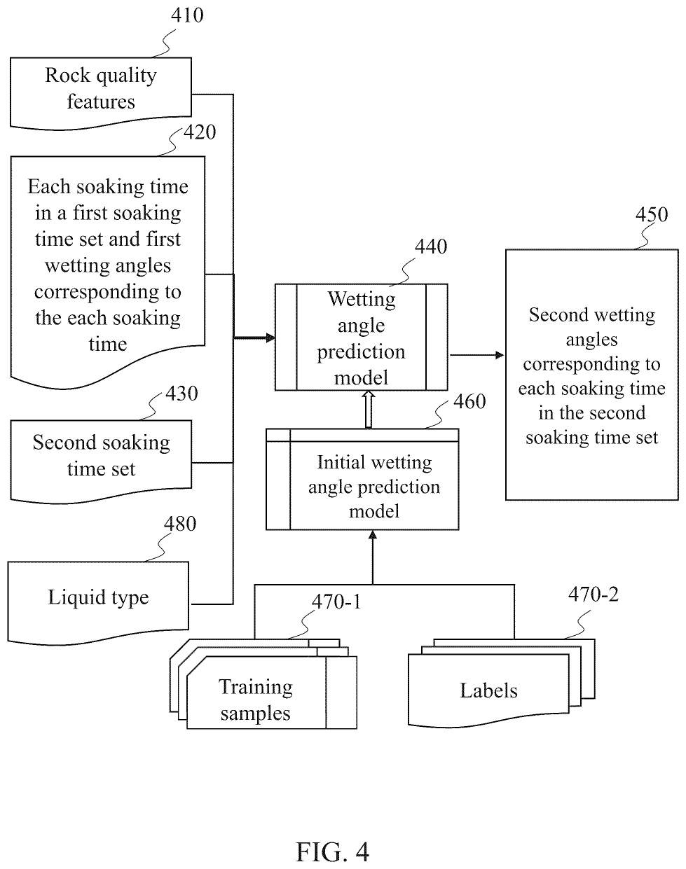

In some embodiments, the calculation module may process the first wetting angles through the wetting angle prediction model to obtain the second wetting angle of the standard core. For more information about the wetting angle prediction model, please refer to FIG. 4 and its related description.

Step S14, obtaining the spontaneous imbibition depth-soaking time curve by utilizing the modified LW model under the spontaneous imbibition effect based on the first wetting angles and the second wetting angles.

In some embodiments, in order to enhance the accuracy of the spontaneous imbibition depth-soaking time curve, a volume of data of the spontaneous imbibition distance of the standard cores at different soaking times by utilizing the modified LW model under the spontaneous imbibition effect may be no less than a preset volume, e.g., no less than thousands of items.

In some embodiments, the soaking time in step S2 may include soaking times in the first soaking time set and the second soaking time set.

In some embodiments, the calculation module may obtain the spontaneous imbibition depth-soaking time curve based on the wetting angle, soaking time, and fluid viscosity, and upload the spontaneous imbibition depth-soaking time curve to the storage device for storage, or transfer the spontaneous imbibition depth-soaking time curve to the terminal device to display to the user through the network.

In some embodiments of the present disclosure, based on the calculation module predicting the second wetting angle through the first wetting angles, and finally obtaining the spontaneous imbibition depth-soaking time curve, this manner shortens a determination process and calculation amount of the wetting angle, and at the same time ensures the accuracy of the spontaneous imbibition depth-soaking time curve and improves higher efficiency, and this manner also processes a large amount of wetting angle data, so as to further improve the accuracy of the spontaneous imbibition depth-soaking time curve.

FIG. 4 is a schematic diagram illustrating an exemplary wetting angle prediction model according to some embodiments of the present disclosure.

The wetting angle prediction model may be a machine learning model for determining a wetting angle. The wetting angle prediction model may be Neural Networks (NN) model or other models. For example, Recurrent Neural Network (RNN) model, etc.

In some embodiments, an input of the wetting angle prediction model 440 may include rock quality features 410, each soaking time in the first soaking time set and first wetting angles 420 corresponding to each soaking time, a second soaking time set 430 and a liquid type 480; an output of the wetting angle prediction model 440 may include second wetting angles 450 corresponding to each soaking time in the second soaking time set. For more information about the first soaking time set, the second soaking time set, and the rock quality feature, please refer to FIG. 3 and its related descriptions.

The liquid type refers to a type of liquid used to soak the standard core. For example, the liquid type may include, but is not limited to, pure water, slick water, etc.

In some embodiments, the calculation module may obtain no less than a preset count of training samples 470-1 with labels 470-2, the training samples 470-1 include sample rock quality features of sample standard cores, a sample first soaking time set, a sample first wetting angle corresponding to the sample first soaking time set, a sample second soaking time set, and a liquid type of sample liquid for soaking sample standard core; and the labels 470-2 of the training samples 470-1 include sample second wetting angles corresponding to the sample second soaking time set. The calculation module may iteratively update an initial wetting angle prediction model 460 by using the no less than a preset count of training samples 470-1 with labels 470-2 to obtain the wetting angle prediction model 440.

For example, the calculation module may input the no less than a preset count of training samples 470-1 into the initial wetting angle prediction model 460, construct a loss function through the labels 470-2 and a result of the initial wetting angle prediction model 460, and iteratively update parameters of the initial wetting angle prediction model 460 based on the loss function. When the loss function of the initial wetting angle prediction model 460 meets a preset iteration condition, the model training is completed, and the trained wetting angle prediction model 440 is obtained. The preset iteration condition may be that the loss function converges, a count of iterations reaches a threshold or the like. For example, the count of iterations may be not less than a preset count, such as not less than 100,000 times. The first soaking time set and the second soaking time set may include a plurality of soaking times.

In some embodiments, the calculation module may obtain training samples and labels in a plurality of ways. For example, the calculation module may obtain training samples and labels through historical data. A count of training samples and labels is not less than a preset count, such as not less than 100,000 sets.

In some embodiments, the calculation module my obtain the training samples and labels based on historical data, and upload the obtained training samples and labels to the storage device for storage through the network.

In some embodiments of the present disclosure, through the wetting angle prediction model to process the rock quality feature, each soaking time of the first soaking time set and a first wetting angle corresponding to the each soaking time set to determine a second wetting angle corresponding to each soaking time in the second soaking time set, this manner can consider various factors simultaneously, so that the determination of the second wetting angle is efficient and accurate, and errors in manual determination can be avoided.

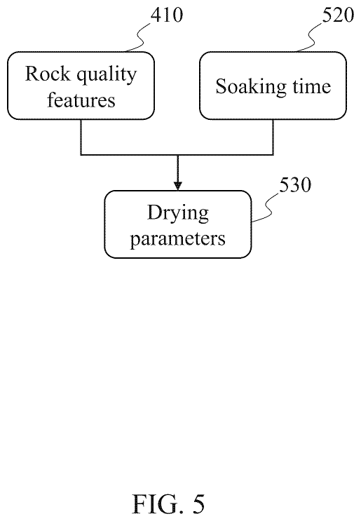

FIG. 5 is a schematic diagram illustrating an exemplary determination of drying parameters according to some embodiments of the present disclosure.

In some embodiments, in step S2, before performing a nano-indentation test on the standard cores, it is necessary to dry the standard cores based on drying parameters. As shown in FIG. 5, drying parameters 530 of the standard cores after the soaking time is determined based on the rock quality features 410 and the soaking time 520 of the standard cores. For more information about the rock quality features, please refer to FIG. 2 and its related descriptions.

The drying parameters refer to parameters related to drying the standard cores. For example, drying parameters may include but are not limited to, a drying temperature, a drying time, or the like.

In some embodiments, the calculation module may determine the drying parameters of the standard cores after the soaking time based on the rock quality feature and soaking time of the standard core in a plurality of ways. For example, the calculation module may determine the drying parameters through a preset data comparison table based on the rock quality features and soaking time of the standard cores. The preset data comparison table records drying parameters corresponding to different rock quality features and soaking times of the standard cores. The preset data comparison table may be preset based on prior knowledge or historical data.

In some embodiments, the drying parameters may be determined based on optimal historical drying parameters obtained through vector matching, and the optimal historical drying parameters are determined based on a modulus similarity threshold. For example, the calculation module may construct a third target vector based on the rock quality features and soaking time; determine a third correlation vector through a third vector database based on the third target vector; and determine reference drying parameters corresponding to the third correlation vector as drying parameters corresponding to the third target vector.

The third target vector refers to a vector constructed based on the rock quality feature and soaking time. For a construction manner of the third target vector, please refer to the above-mentioned construction manner of the first target vector in FIG. 3.

The third vector database includes a plurality of third reference vectors, and each third reference vector of the plurality of third reference vectors has corresponding reference drying parameters. For example, a count of the third reference vector in the third vector database may be not less than a preset count, such as not less than 100,000.

The third reference vector refers to a vector constructed based on a historical rock quality feature and a historical soaking time when the standard cores are dried during a historical time period. The reference drying parameters corresponding to a reference vector may be historical optimal drying parameters when the standard cores are dried during a historical time period. For a construction manner of the third reference vector, please refer to the construction manner of the first target vector in FIG. 3.

In some embodiments, the calculation module may calculate a vector distance between the third target vector and the third reference vector, and determine the drying parameters of the third target vector. For a manner to determine the drying parameters of the third target vector, please refer to the manner to determine the equivalent soaking condition of the first target vector in FIG. 3.

In some embodiments, the historical optimal drying parameters may be determined in the following manner. In a real environment, a sample core with a rock quality feature corresponding to the third reference vector is obtained, and under a soaking condition corresponding to the third reference vector, and then after being naturally dried in an internal environment of rock layer (for example, placing the sample core in a real environment of rock layer for drying), sample Young's modulus of the sample core is obtained through performing a test on the sample core and calculation; a plurality of sets of candidate drying parameters are generated (e.g., randomly generating); for each set of candidate drying parameters, the sample core is simulated drying (for example, drying by a simulated drying software), and Young's modulus of the sample core is calculated after drying based on the set of candidate drying parameters; a similarity between Young's modulus corresponding to the candidate drying parameters and the sample Young's modulus is calculated, and candidate drying parameters whose similarity is greater than a modulus similarity threshold are determined as the historical optimal drying parameters. The modulus similarity threshold may be a default value, a default value, etc.

In some embodiments, if there is a plurality of candidate drying parameters whose similarity is greater than the modulus similarity threshold, candidate drying parameters with the shortest drying time may be determined as the historical optimal drying parameters, which is conducive to shortening a drying time and improving efficiency.

In some embodiments, the modulus similarity threshold may be related to core parameter sensitivity. For example, the greater the core parameter sensitivity is, the smaller the modulus similarity threshold may be set.

The core parameter sensitivity refers to a change degree of parameters of a core after the core is processed under different conditions. For example, the core parameter sensitivity may be used to reflect a change degree of parameters for evaluating rock properties such as the wetting angle and Young's modulus of the core after soaking, heating, pressurizing, and other treatments. The core parameter sensitivity may be represented by a real number between 0 and 1. The larger the number is, the greater the change degree of parameters of a core is after the core is processed under different conditions.

In some embodiments, the calculation module may determine the core parameter sensitivity in a plurality of ways. For example, the calculation module may construct a fourth target vector based on the rock quality feature; determine a fourth correlation vector through a fourth vector database based on the fourth target vector; and determine a reference core parameter sensitivity corresponding to the fourth correlation vector as a core parameter sensitivity corresponding to the fourth target vector. For more information about the rock quality feature, please refer to FIG. 3 and its related descriptions.

The fourth target vector refers to a vector constructed based on the rock quality feature. For a construction manner of the fourth target vector, please refer to the construction manner of the first target vector in FIG. 3.

The fourth vector database includes a plurality of fourth reference vectors, and each fourth reference vector in the plurality of fourth reference vectors has a corresponding reference core parameter sensitivity. For example, a count of fourth reference vectors in the fourth vector database may be not less than a preset count, such as not less than 100,000.

The fourth reference vector refers to a vector constructed based on the historical rock quality feature of the core during a historical time period. For a construction manner of the fourth reference vector, please refer to the construction manner of the first target vector in FIG. 3.

The core parameter sensitivity corresponding to the fourth reference vector may be determined in the following manner. A large number of tests are conducted on cores with different rock quality features, after being processed under different conditions (for example, soaking, heating, pressurizing), parameters for evaluating rock properties such as a wetting angle, Young's modulus of a core with a rock quality feature change greatly (for example, greater than a change degree threshold), then the core parameter sensitivity of the core with the rock quality feature is higher. In some embodiments, a conversion ratio, conversion equation, etc. may also be preset to convert a change degree of parameters for evaluating rock properties such as a wetting angle and Young's modulus of the core after a test into the core parameter sensitivity of the core with the rock quality feature.

In some embodiments of the present disclosure, by correlating the modulus similarity threshold with the core parameter sensitivity and considering a change degree of a wetting angle, Young's modulus, etc. of cores with different rock quality features after processing, the modulus similarity threshold is set more reasonably, which is conducive to obtaining more optimal drying parameters.

In some embodiments, the calculation module may determine the drying parameters based on the rock quality feature and soaking time, and upload the determined drying parameters to the storage device for storage through the network.

In some embodiments of the present disclosure, drying the standard cores based on the drying parameters realizes a more accurate simulation of a drying condition in a real environment, which is conducive to improving the accuracy of the Young's modulus-soaking time curve. Based on the determination of drying parameters by the calculation module, a large number of candidate drying parameters may be generated and simulated to obtain drying conditions that are closer to the real environment, further improving the accuracy of the subsequent obtained Young's modulus-soaking time curve.

In some embodiments, in response to receiving calculation instruction, the formation parameters are input into the calculation formula for the proppant embedded volume to obtain the predicted proppant embedded volume. The calculation formula for the proppant embedded volume is determined by a process including the following steps.

Step S1, determining a spontaneous imbibition depth-soaking time curve. The spontaneous imbibition depth-soaking time curve may be obtained by making a target block shale into five standard cores, soaking the five standard cores for 0, 3, 5, 7, and 15 days respectively, then conducting a spontaneous imbibition test on faces of the five standard cores at different soaking times, and utilizing a modified LW model under a spontaneous imbibition effect.

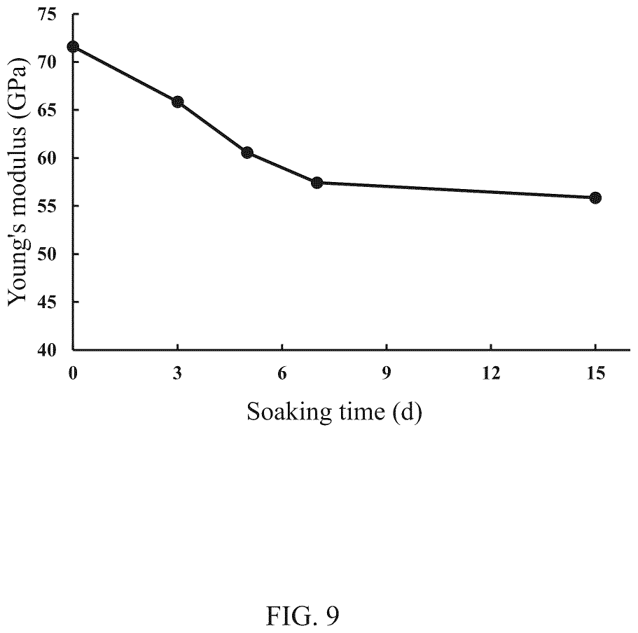

Step S2, determining a Young's modulus-soaking time curve of the standard cores. The Young's modulus-soaking time curve of the standard cores may be obtained by conducting a nano-indentation test on upper and lower faces of the cores after drying the five standard cores in step S1, as shown in FIG. 9.

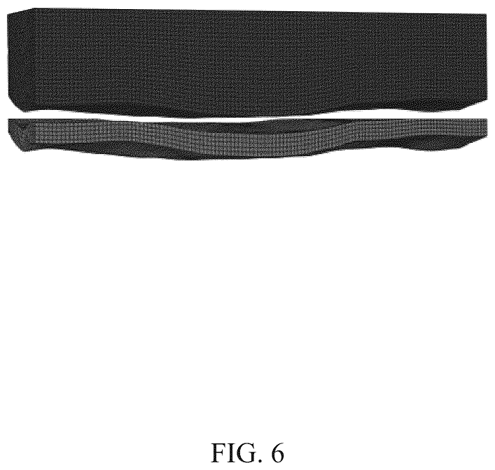

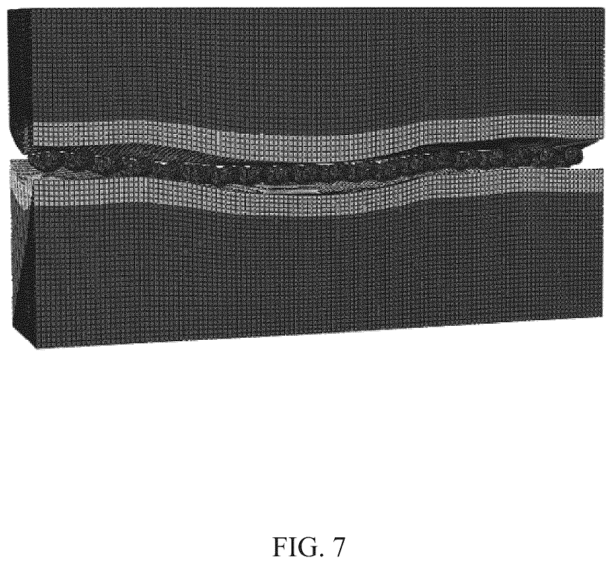

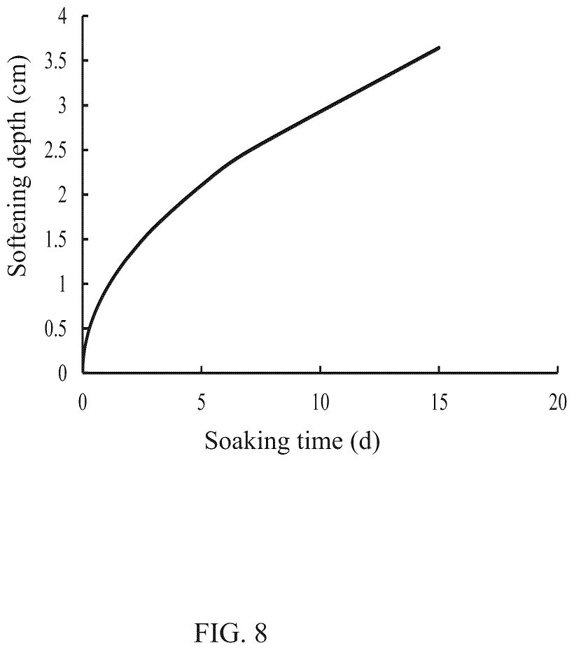

Step S3, establishing a 3D model of proppant embedded in a rock slab by a finite element manner, wherein the rock slab in the 3D model is divided into an unsoftened layer and a softened layer (as shown in FIG. 6), and Young's modulus of the unsoftened layer is set as Young's modulus of the standard cores; softening depth of the softened layer is set as a softening depth-soaking time curve (as shown in FIG. 8) according to the spontaneous imbibition depth-soaking time curve, Young's modulus of the softened layer is set according to the Young's modulus-soaking time curve, and a proppant embedding model containing the softened layer (as shown in FIG. 7) is obtained, wherein parameters of the softened layer of the proppant embedding model are as shown in Table 1, and basic parameters of the rock slab and proppant are shown in Table 2.

TABLE 1

| | Thickness of | Young's modulus of | Thickness of | Young's modulus |
|---|---|---|---|---|
| Soaking | unsoftened | unsoftened layer | softened | of softened layer |
| time (day) | layer (mm) | (GPa) | layer (mm) | (GPa) |
| 0 | 50 | 72 | 0 | 0 |
| 3 | 34 | 72 | 16 | 65 |
| 5 | 29 | 72 | 21 | 60 |
| 7 | 25 | 72 | 25 | 56 |
| 15 | 14 | 72 | 36 | 54 |

TABLE 2

| Length of rock | Width of rock | Thickness of | Poisson's ratio |
|---|---|---|---|
| slab (mm) | slab (mm) | rock slab (mm) | of rock slab |
| 129 | 3 | 50 | 0.12 |
| | | Number of | |
| Particle size of | Young's modulus | laying layers | Poisson's ratio |
| proppant (mm) | of proppant (GPa) | (layer) | of proppant |
| 0.2 | 13 | 1 | 0.2 |

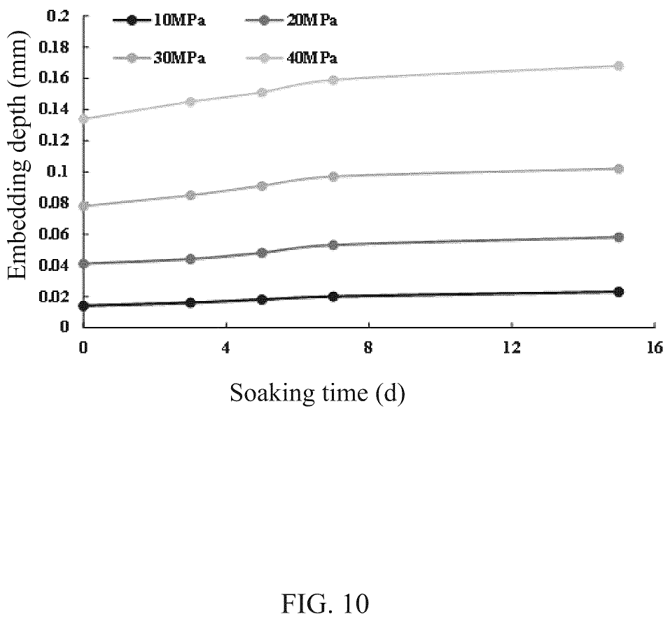

Step S4, obtaining an embedding volume-soaking time curve (embedding depth in FIG. 10 is the embedding volume) by performing numerical simulation on the proppant embedding model containing the softened layer according to data in Table 1 and Table 2, including: according to the different soaking times, setting simulated parameters of the proppant embedding model containing the softened layer respectively; applying closure stress to an upper slab of the 3D model by utilizing a stress interaction effect, completely fixing a lower slab of the 3D model to simulate a crustal fracture closure process, outputting an average embedding volume of the upper slab and lower slab after the model is stabilized, and obtaining the embedding volume-soaking time curve at the different soaking times.

Step S5, obtaining an embedding volume at a soaking time ti according to the embedding volume-soaking time curve obtained by the numerical simulation, and introducing the embedding volume into the calculation formula for the proppant embedding volume of the proppant embedded in a rock mass, and obtaining equivalent Young's modulus Eₜ₁ at the soaking time t₁ by calculating reversely, repeating the above process, obtaining equivalent Young's modulus Eₜ₂ at a soaking time t₂, equivalent Young's modulus Eₜ₃ at a soaking time t₃, . . . , equivalent Young's modulus Eₜₙ at a soaking time tₙ.

Step S6, according to equivalent Young's modulus corresponding to the different soaking times, obtaining equivalent Young's modulus of a softened rock slab by regression, wherein Eₜ is a function related to a soaking time, a relationship formula of which is as follows:

Eₜ=0.968E₀e^{-018t}.

Then obtaining a calculation formula for a proppant embedding volume considering the softening effect:

$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{t} \}$$

$${E}_{t} = a {E}_{0} {e}^{- bt} .$$

Where w denotes the proppant embedding volume, a unit of which is mm; a₀ and a₁ denote modification factors, which are 0.0646 and 18.2 respectively, and dimensionless; R denotes the a particle size of proppant, a unit of which is mm; P denotes the crustal stress, a unit of which is MPa; E₁ denotes the Young's modulus of the proppant, a unit of which is MPa; v₁ denotes the Poisson's ratio of the proppant, which is dimensionless; v₂ denotes the Poisson's ratio of the rock slab, which is dimensionless; H denotes the thickness of the rock slab, a unit of which is mm; t denotes the soaking time, a unit of which is d; Eₜ denotes the equivalent Young's modulus, a unit of which is MPa; and Eo denotes the Young's modulus of the standard core, a unit of which is MPa.

The basic concepts have been described above, obviously, for those skilled in the art, the above-detailed disclosure is only an example and does not constitute a limitation to the present disclosure. Although not expressly stated here, those skilled in the art may make various modifications, improvements, and corrections to the present disclosure. Such modifications, improvements, and corrections are suggested to the present disclosure, so such modifications, improvements, and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure, or characteristic related to at least one embodiment of the present disclosure. Therefore, it should be emphasized and noted that two or more references to “one embodiment” “an embodiment” or “an alternative embodiment” in different places in the present disclosure do not necessarily refer to the same embodiment. In addition, certain features, structures, or characteristics in one or more embodiments of the present disclosure may be properly combined.

In addition, unless explicitly stated in the claims, the order of processing elements and sequences described in the present disclosure, the use of numbers and letters, or the use of other names are not used to limit the sequence of processes and methods in the present disclosure. While the foregoing disclosure has discussed some embodiments of the invention that are presently believed to be useful by way of various examples, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in the present disclosure and help the understanding of one or more embodiments of the present disclosure, in the foregoing description of the embodiments of the present disclosure, sometimes multiple features are combined into one embodiment, drawings, or descriptions thereof. This method of disclosure does not, however, imply that the subject matter of the present disclosure requires more features than are recited in the claims. Rather, claimed subject matter may lie in less than all features of a single foregoing disclosed embodiment.

In some embodiments, numbers describing the number of components and attributes are used, and it should be understood that such numbers used in the present disclosure of the embodiments, in some examples, use the modifiers “about”, “approximately” or “substantially”. Unless otherwise stated, “about”, “approximately” or “substantially” indicates that the stated figure allows for a variation of ±20%. Accordingly, in some embodiments, the numerical parameters used in the present disclosure and claims are approximations that can vary depending on the desired characteristics of individual embodiments. In some embodiments, numerical parameters should take into account the specified significant digits and adopt the general digit reservation method. Although the numerical ranges and parameters used in some embodiments of the present disclosure to confirm the breadth of the range are approximations, in specific embodiments, such numerical values should be set as precisely as practicable.

Each patent, patent application, patent application publication, and other material, such as article, book, specification, publication, document, etc., cited in the present disclosure is hereby incorporated by reference in its entirety. Historical application documents that are inconsistent with or conflict with the content of the present disclosure are excluded, and documents (currently or later appended to the present disclosure) that limit the broadest scope of the claims of the present disclosure are excluded. It should be noted that if there is any inconsistency or conflict between the descriptions, definitions, and/or terms used in the accompanying materials of the present disclosure and the contents of the present disclosure, the descriptions, definitions, and/or terms used in the present disclosure shall prevail.

Finally, it should be understood that the embodiments described in the present disclosure are only used to illustrate the principles of the embodiments of the present disclosure. Other modifications are also possible within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A calculation system for predicting a proppant embedding depth based on a shale softening effect, comprising: a sampling test terminal, a scheduling module, a monitoring module, and a calculation module, wherein the scheduling module, the monitoring module, and the calculation module are connected in communication, and the monitoring module is connected to an external operating system through a wireless network, wherein
the external operating system is configured to perform a hydraulic fracturing operation and receive a first control signal or a second control signal from the monitoring module;
the sampling test terminal is configured to:
in response to receiving a sampling test instruction, take one or more samples from a rock layer, test the samples, and obtain test data;
the scheduling module is configured to:
obtain one or more sets of candidate construction parameters, the candidate construction parameters including candidate proppant parameters and candidate operation areas;
obtain formation parameters of the candidate operation areas among the candidate construction parameters;
based on the candidate construction parameters and the formation parameters of the candidate operation area, generate a calculation instruction, send the calculation instruction to the calculation module, and obtain predicted proppant embedding volumes corresponding to the candidate construction parameters from the calculation module; and
determine a target construction parameter from the candidate construction parameters whose the predicted proppant embedding volumes are less than first preset thresholds;
the monitoring module is configured to:
obtain the target construction parameters for mining, and obtain the predicted proppant embedding volume corresponding to the target construction parameter from the calculation module;
determine a hydraulic fracture warning condition during an operation period based on the predicted proppant embedded volume corresponding to the target construction parameter;
for each preset cycle,
determine characteristic parameters of one or more hydraulic fractures and rock quality features of one or more points in a target operation area based on second sensing data obtained at multiple times from one or more sensors deployed in the target operation area;
determine, based on the rock quality features, actual estimated proppant embedding volumes corresponding to the rock quality features of one or more points in the target operation area through the test data under current proppant parameters;
based on a difference between the actual estimated proppant embedding volumes and a preset proppant embedding volume in the target operation area, issue a first warning message and/or send the first control signal to a fracturing control pump in the external operating system, wherein the first control signal indicates that the fracturing control pump performs an adjustment on a particle size of proppant or a concentration of proppant of the fracturing fluid injected into a pipeline, and an adjustment amount of the adjustment in the first control signal is determined based on the difference between the actual estimated proppant embedding volumes and the preset proppant embedding volume; and
in response to the characteristic parameters of the one or more hydraulic fractures meeting the hydraulic fracture warning condition, issue a second warning information and/or send the second control signal to the fracturing control pump in the external operation system, wherein the second control signal indicates that the fracturing control pump performs an adjustment on the particle size of the proppant or the concentration of the proppant of the fracturing fluid into the pipeline, and an adjustment amount of the adjustment in the second control signal is determined based on a difference between the characteristic parameters of the one or more hydraulic fractures and a threshold in the hydraulic fracture warning condition.

2. The calculation system of claim 1, wherein the calculation module is configured to:
in response to receiving the calculation instruction, obtain the predicted proppant embedding volumes by inputting the formation parameters into a calculation formula for a proppant embedding volume, wherein the calculation formula for the proppant embedding volume is determined by a process including:
step S1, determining a spontaneous imbibition depth-soaking time curve, wherein the spontaneous imbibition depth-soaking time curve is obtained by conducting a spontaneous imbibition test on faces of different standard cores at different soaking times, respectively and utilizing a modified Lucas-Washburn (LW) model under a spontaneous imbibition effect, and the standard cores are obtained based on a target block shale;
step S2, determining a Young's modulus-soaking time curve of core surfaces, wherein the Young's modulus-soaking time curve is obtained by drying the standard cores at the different soaking times and conducting a nano-indentation test on surfaces of the standard cores, respectively;
step S3, establishing a 3D model of proppant embedded in a rock slab by a finite element manner, wherein the rock slab in the 3D model is divided into an unsoftened layer and a softened layer, and Young's modulus of the unsoftened layer is set as Young's modulus of the standard cores; a thickness of the softened layer is set according to the spontaneous imbibition depth-soaking time curve, and Young's modulus of the softened layer is set according to the Young's modulus-soaking time curve, and a proppant embedding model containing the softened layer is obtained;
step S4, obtaining an embedding volume-soaking time curve by performing numerical simulation on the proppant embedding model containing the softened layer with set parameters; wherein a process of the numerical simulation in the step S4 includes:
setting simulated parameters of the proppant embedding model containing the softened layer respectively according to the different soaking times;
applying closure stress to the unsoftened layer of the 3D model by utilizing a stress interaction effect, and fixing the softened layer of the 3D model to simulate a crustal fracture closure process, outputting an average embedding volume of the unsoftened layer and the softened layer after the 3D model is stabilized, and obtaining the embedding volume-soaking time curve at the different soaking times; and
step S5, modifying equivalent Young's modulus of proppant embedded in a rock mass based on the embedding volume-soaking time curve, and obtaining the calculation formula for proppant embedding volume considering the softening effect;
$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{t} \}$$
$${E}_{t} = a {E}_{0} {e}^{- bt}$$
where w denotes a proppant embedding volume, a unit of which is millimeter (mm); a₀ and a₁ denote modification factors, which are 0.0646 and 18.2 respectively, and dimensionless; R denotes a particle size of proppant, a unit of which is mm; P denotes crustal stress, a unit of which is MegaPascal (MPa); E₁ denotes Young's modulus of the proppant, a unit of which is MPa;
denotes a Poisson's ratio of the proppant, which is dimensionless; v₂ denotes a Poisson's ratio of the rock slab, which is dimensionless; H denotes a thickness of a rock slab, a unit of which is mm; t denotes a soaking time, a unit of which is day (d); Eₜ denotes an equivalent Young's modulus, a unit of which is MPa; E₀ denotes Young's modulus of a standard core, a unit of which is MPa; and a and b denote fitting coefficients;
wherein the modifying equivalent Young's modulus of proppant embedded in a rock mass based on the embedding volume-soaking time curve in the step S5 includes:
(1) obtaining an embedding volume at a soaking time t₁ according to the embedding volume-soaking time curve obtained by the numerical simulation, and introducing the embedding volume into the calculation formula for the proppant embedding volume of the proppant embedded in the rock mass, and obtaining equivalent Young's modulus Eₜ₁ at the soaking time t₁ by calculating reversely, similarly, obtaining equivalent Young's modulus Eₜ₂ at a soaking time t₂, equivalent Young's modulus Eₜ₃ at a soaking time t₃, . . . , equivalent Young's modulus Eₜₙ at a soaking time tn, wherein tn refers to any time of soaking time, and n is an integer and n≥3; and
(2) according to the equivalent Young's modulus corresponding to the different soaking times, obtaining the equivalent Young's modulus of a softened rock slab by regression.

3. The calculation system of claim 1, wherein different candidate construction parameters corresponding to different first preset thresholds.

4. The calculation system of claim 3, wherein the first preset thresholds corresponding to the candidate construction parameters are determined by a process including:
determining the candidate operation areas corresponding to the candidate construction parameters; and
determining the first preset thresholds corresponding to the candidate construction parameters based on a dispersion degree of formation characteristics of a plurality of points obtained by sensors in the candidate operation areas.

5. The calculation system of claim 1, wherein to obtain formation parameters of the candidate operation areas among the candidate construction parameters, the scheduling module is further configured to:
determine an average formation stress of the candidate operation areas based on first sensing data obtained at a plurality of times by one or more sensors deployed in the candidate operation areas; and
generate a sampling test instruction, send the sampling test instruction to the sampling test terminal, and determine the formation parameters of the candidate operation areas based on the test data obtained from the sampling test terminal.

6. The calculation system of claim 1, wherein the preset period is related to at least one of a size of the target operation area, a total depth of formation, and a remaining available computing resource.

7. The calculation system of claim 1, wherein the candidate proppant parameters include at least one of a particle size of a candidate proppant, a concentration of the candidate proppant, a Young's modulus of the candidate proppant, and a Poisson's ratio of the candidate proppant.

8. The calculation system of claim 1, wherein the formation parameters include at least one of a thickness of the rock layer, a Poisson's ratio of the rock layer, and a Young's modulus of the rock layer.

9. The calculation system of claim 1, wherein the characteristic parameters of the one or more hydraulic fractures include at least one of a length of the one or more hydraulic fractures, a width of the one or more hydraulic fractures, and an effective support area of the one or more hydraulic fractures.

10. The calculation system according to claim 2, wherein the Step 51 further includes:
step S11, determining a first soaking time set;
step S12, determining first wetting angles of the standard cores, wherein the first wetting angles include wetting angles of the standard cores corresponding to the at least one soaking time in the first soaking time set;
step S13, predicting second wetting angles of the standard cores based on the first wetting angles; wherein the second wetting angles include wetting angles of the standard cores corresponding to the least one soaking time in a second soaking time set; and
step S14, obtaining the spontaneous imbibition depth-soaking time curve by utilizing the modified LW model under the spontaneous imbibition effect based on the first wetting angles and the second wetting angles.

11. The calculation system according to claim 10, wherein the different soaking times in the step S2 includes the soaking time in the first soaking time set and the second soaking time set.

12. The calculation system according to claim 10, wherein the predicting second wetting angles of the standard cores based on the first wetting angles includes:
determining the second wetting angles of the standard cores through processing the first wetting angles by a wetting angle prediction model, wherein the wetting angle prediction model is a machine learning model.

13. The calculation system according to claim 12, wherein the wetting angle prediction model is obtained by a training process including:
obtaining training samples with labels, whose count is no less than a preset count, wherein the training samples include sample rock quality features of sample standard cores, a sample first soaking time set, first sample wetting angles corresponding to the sample first soaking time set, a sample second soaking time set, and a liquid type of sample liquid for soaking sample standard cores; the labels of the training samples are second sample wetting angles corresponding to the second sample soaking time set; and
iteratively updating an initial wetting angle prediction model by utilizing the training samples with labels to obtain the wetting angle prediction model.

14. The calculation system according to claim 10, wherein the determining first wetting angles of the standard cores includes:
determining a corresponding equivalent soaking condition based on the soaking time in the first soaking time set, conducting a soaking test on the standard cores with the equivalent soaking condition, and determining wetting angles obtained from a test result as the first wetting angles of the standard cores.

15. The calculation system according to claim 14, wherein the determining a corresponding equivalent soaking condition based on the soaking time in the first soaking time set includes:
determining the equivalent soaking condition corresponding to the soaking time through processing the soaking time in the first soaking time set by an equivalent soaking condition determination model, wherein the equivalent soaking condition determination model is a machine learning model.

16. The calculation system according to claim 2, wherein a relationship formula of the modified LW model under the spontaneous imbibition effect in the step S1 is:
h(t)=\sqrt{(rδγt cos θ)/2 τμ}.
where h(t) denotes a spontaneous imbibition distance, a unit of which is m; t denotes the soaking time, a unit of which is second (s); r denotes an equivalent capillary radius, a unit of which is meter (m); γ denotes a fluid interfacial tension, a unit of which is Newton/meter (N/m); δ denotes a pore-shape factor, which is dimensionless; θ denotes a wetting angle, a unit of which is °; τ denotes a pore tortuosity, which is dimensionless; and μ denotes a fluid viscosity, a unit of which is Pascal second (Pa·s).

17. The calculation system according to claim 2, wherein the drying the standard cores in the step S2 is performed based on drying parameters, the drying parameters include a drying temperature and a drying time, and the drying parameters are determined by a process including:
determining the drying parameters of the standard cores under the different soaking times based on rock quality features and the different soaking times of the standard cores.

18. The calculation system according to claim 17, wherein the drying parameters are determined based on optimal historical drying parameters obtained by vector matching, the optimal historical drying parameters are determined based on a modulus similarity threshold, and the modulus similarity threshold is related to a core parameter sensitivity.

19. The calculation system according to claim 2, wherein a sum of the thickness of the unsoftened layer and the thickness of the softened layer in the step S3 is equal to an overall thickness of the rock slab.

20. The calculation system according to claim 2, wherein the calculation formula for the proppant embedding volume of the proppant embedded in the rock mass is:
$$w = {a}_{0} + {a}_{1} \{ 1.04 {R ( P )}^{2 / 3} [ {( \frac{1 - {v}_{1}^{2}}{{E}_{1}} + \frac{1 - {v}_{2}^{2}}{{E}_{t}} )}^{2 / 3} - {( \frac{1 - {v}_{1}^{2}}{{E}_{1}} )}^{2 / 3} ] + HP / {E}_{2} \}$$
where w denotes the proppant embedding volume, a unit of which is mm; a₀ and a₁ denote modification factors, which are 0.0646 and 18.2 respectively, and dimensionless; R denotes the particle size of the proppant, a unit of which is mm; P denotes the crustal stress, a unit of which is MPa; E₁ denotes the Young's modulus of proppant, a unit of which is MPa; E₂ denotes the Young's modulus of the rock slab, a unit of which is Giga pascal (GPa); denotes the Poisson's ratio of the proppant, which is dimensionless; v₂ denotes the Poisson's ratio of the rock slab, which is dimensionless; and H denotes the thickness of the rock slab, a unit of which is mm.
